(19) United States
(12) Patent Application Publication
(10) Pub. No.: US 2002/0077512 A1
(43) Pub. Date: Jun. 20, 2002

(54) Hydrocarbon conversion system and method with a plurality of sources of compressed oxygen-containing gas

(76) Inventors: Rex Carl Tendick, Tulsa, OK (US); John Timothy O'Beck, Tulsa, OK (US); Mathew R. Nimmo, Bartlesville, OK (US); Tsungani Yana Francis Record, Tulsa, OK (US)

Correspondence Address: LAW OFFICES OF GRADY K. BERGEN, 2626 COLE AVENUE, SUITE 400, DALLAS, TX, 75204, US

(21) Appl. No.: 10/011,789

(22) Filed: Dec. 5, 2001

Related U.S. Application Data
(60) Provisional application No. 60/257,752, filed on Dec. 20, 2000.

(51) Int. Cl. C07C 27/00

(52) U.S. Cl. 568/959

(57) ABSTRACT

A system and method for converting light hydrocarbons into heavier hydrocarbons, such as with a Fischer-Tropsch process, are provided that use a gas turbine and that uses at least two different sources of compressed oxygen-containing gas for the preparation of synthesis gas. The system and method may also include a steam turbine powered by process steam, along with the gas turbine, to provide additional power to produce the compressed oxygen containing gas.

### BACKGROUND

A need has existed for a long time for a process to convert solid carbonaceous and light or gaseous hydrocarbon materials to liquid fuels and other useful products. One successful approach to meeting this need is to first gasify solid carbonaceous materials and then to synthetically convert light hydrocarbons into heavier hydrocarbons through the Fischer-Tropsch (F-T) process. The synthetic production of hydrocarbons by the catalytic reaction of synthesis gas is well known and is generally referred to as the Fischer-Tropsch reaction. This process was developed in early part of the 20^{th} century in Germany. It has been practiced commercially in Germany during World War II and later in South Africa.

Fischer-Tropsch hydrocarbon conversion systems typically have a synthesis gas generator and a Fischer-Tropsch reactor unit. The synthesis gas generator receives unconverted hydrocarbons, such as light hydrocarbons found in natural gas, and produces synthesis gas. The synthesis gas is then delivered to a Fischer-Tropsch reactor. In the F-T reactor, the synthesis gas is converted to heavier, longer-chain hydrocarbons. Recent examples of Fischer-Tropsch systems include U.S. Pat. Nos. 4,883,170; 4,973,453; 5,733,941; and 5,861,441 all of which are incorporated by reference herein for all purposes.

While Fischer-Tropsch processes offer great environmental benefits compared to other sources of energy, the plants must be relatively economic before wide-scale adoption will occur. One avenue to seek increased efficiencies on such systems has been with respect to the integration of turbines. Gas turbines are typically comprised of a combustor, expander and compressor. Fuel along with compressed air or an oxygen-containing gas stream is combusted within the combustor of the turbine. The combustion gases from the combustor are introduced into an expander, which powers the compressor for supplying the compressed air or oxygen for the combustion reaction.

One example of the use of a gas turbine in such systems is disclosed in U.S. Pat. No. 5,733,941, which is incorporated by reference herein for all purposes. The '941 patent presents a conversion system that incorporates a Brayton cycle having a combustor and power turbines. A low-BTU tail gas is brought back from the Fischer-Tropsch conversion unit to the combustor of the turbine. The compressor(s) of the turbine in the '941 patent is used to compress all of the air that is delivered to an autothermal reformer used in the conversion process. Efforts to incorporate gas turbines to efficiently compress air have been suggested in other areas as well. For example, U.S. Pat. No. 5,177,114, which primarily presents a methanol system, shows a gas turbine used in series with a compressor to compress air that is used to produce synthesis gas.

One of the problems associated with utilizing gas turbines in conversion processes, particularly in GTL processes for converting light hydrocarbons (i.e. C₄ or less) to heavier hydrocarbons (C₅+), is that the amount of compressed air that can be diverted from the gas turbine is limited. Diverting an excessive amount of compressed air for use as process air in the conversion process can upset the mass balance of fluid flow to the gas turbine, thus affecting its operation. In many instances, the maximum amount of diverted air may be insufficient to make up the needed process air for the conversion process. Thus, additional compressed air provided by external sources may be required.

It is usually desirable to make use of tail or waste gases that would otherwise be flared off as wasted heat energy. Thus, tail gas has been used as a fuel for powering the gas turbine, such as disclosed in the '941 patent. Fluctuations in the energy value of the tail gases, however, may affect the operation of the gas turbine and thus the amount of compressed air produced. Variations in the amount of compressed air diverted as process air can, in turn, affects the downstream processes, often resulting in lower production. Supplementing the tail gas with externally supplied supplemental fuels may help, but this only adds to the cost of the process, particularly were fuel costs are high, and is therefore undesirable.

What is therefore needed is a hydrocarbon conversion system and/or method, particularly a GTL system or process, where adequate process air or oxygen-containing gas is supplied to the system or process and which makes use of tail gases produced during the process, as well as other energy produced by the process, and without the need for externally supplied energy sources.

### SUMMARY

A method for converting hydrocarbons in a hydrocarbon conversion system is provided. The method includes delivering unconverted hydrocarbons to a hydrocarbon conversion unit, which may include a Fischer-Tropsch reactor. Hydrocarbon products and tail gas are produced from the unconverted hydrocarbons utilizing the conversion unit. A gas turbine is also provided having a combustor, an expander and a compressor. Tail gas is delivered to the gas turbine as fuel to fuel the combustor and power the turbine. A first compressed oxygen-containing gas stream, such as air, is produced from the compressor of the gas turbine. At least one other compressor, such as an axial compressor, is provided that is coupled to and powered at least in part by the gas turbine to produce a second compressed oxygen-containing gas stream. From about 50% to 100% of the first compressed oxygen-containing gas stream is delivered to the combustor and expander. The remainder of from 0% to about 50% by volume of the first compressed oxygen-containing gas stream is delivered to the hydrocarbon conversion unit. The second compressed oxygen-containing gas stream is delivered to the hydrocarbon conversion unit in an amount of from about 50% to 100% by volume of that required by the hydrocarbon conversion unit. The first compressor and the at least one other compressor provide all of the compressed oxygen-containing gas required by the conversion unit.

In more specific embodiments, a steam turbine is coupled to the at least one other compressor. Pressurized steam is delivered to the steam turbine to produce power, with at least a portion of the power from the steam turbine being provided to the at least one other compressor. The tail gas may be a low-BTU tail gas, and may be the only fuel delivered to the gas turbine. The unconverted hydrocarbons may be light hydrocarbons of from C₁ to C₄ and the hydrocarbon products may be C₅ or higher.

In still other more specific embodiments, the tail gas provides sufficient mass flow to the gas turbine to compensate for variations in mass flow provided by the first compressed oxygen-containing gas stream delivered to the gas turbine so that power output from the gas turbine is maintained. Additionally, the tail gas may provide sufficient mass flow to the gas turbine so that power output from the gas turbine is above that of the iso nameplate power output rating of the gas turbine at any given ambient temperature or pressure conditions.

In another embodiment of the invention, a hydrocarbon conversion system is provided. The system includes a gas turbine having a combustor, an expander and a compressor for producing a first compressed oxygen-containing gas stream. At least one other compressor, which may be a single-shaft, axial compressor, is provided that is powered at least in part by the gas turbine to produce a second compressed oxygen-containing gas stream. A hydrocarbon conversion unit for converting unconverted hydrocarbons to converted hydrocarbon products and tail gas, which may be a low-BTU tail gas, is also provided with the system. The hydrocarbon conversion unit is in fluid communication with at least one of the turbine compressor and the second compressor for receiving at least one of the first and second compressed oxygen-containing gas streams. The hydrocarbon conversion unit has a tail gas outlet that is in fluid communication with the gas turbine for delivering the tail gas as fuel to the combustor to thereby power the gas turbine.

In more specific embodiments, a steam turbine can be coupled to the at least one other compressor. The steam turbine is in fluid communication with a steam outlet of the hydrocarbon conversion unit and pressurized steam produced during the conversion of the unconverted hydrocarbons is delivered from the conversion unit to the steam turbine through the steam outlet to produce power. At least a portion of the power from the steam turbine is provided to the at least one other compressor.

An electrical motor-generator can also be coupled to the at least one other compressor. The electrical motor-generator is selectively operable as a motor for providing power to the at least one other compressor and as a generator for producing electrical power.

In another more specific embodiment, the hydrocarbon conversion unit is a gas-to-liquid (GTL) conversion unit and includes a syngas reactor containing a catalyst for producing syngas from the unconverted hydrocarbons. A Fischer-Tropsch reactor containing Fischer-Tropsch catalyst for converting the syngas to liquid hydrocarbons is also provided.

In still another embodiment of the invention, a hydrocarbon conversion system is provided having a gas turbine having a combustor, an expander, and a compressor. A steam turbine is also provided. A rotatable shaft is coupled to the steam turbine and the gas turbine, and is rotatably driven by the gas turbine and steam turbine. A hydrocarbon conversion unit for converting unconverted hydrocarbons to converted hydrocarbon products and tail gas is also provided with the system. The hydrocarbon conversion unit has a tail gas outlet that is in fluid communication with the gas turbine for delivering the tail gas as fuel to the combustor to thereby power the gas turbine. The hydrocarbon conversion unit also has a steam outlet in fluid communication with the steam turbine for delivering pressurized steam produced by the hydrocarbon conversion unit during conversion of the unconverted hydrocarbons to the steam turbine to thereby power the steam turbine. At least one or more shaft-driven mechanical devices are coupled to the shaft and driven when the shaft is rotated. A compressed oxygen-containing feed stream conduit is in fluid communication between the hydrocarbon conversion unit and at least one of the compressor and said one or more shaft-driven mechanical devices for delivering compressed air to the hydrocarbon conversion unit.

In another embodiment, a method for converting hydrocarbons is provided. The method includes converting unconverted hydrocarbons to converted hydrocarbon products and tail gas in a hydrocarbon conversion unit and producing pressurized steam as a byproduct of the conversion process. The tail gas is delivered as fuel to a gas turbine having a combustor, an expander, and a compressor for powering the gas turbine. The pressurized steam is delivered to a steam turbine for powering the steam turbine. The gas turbine and steam turbine are coupled to a rotatable shaft that drives at least one shaft-driven mechanical device during operation of the gas and steam turbines. A compressed oxygen-containing gas stream from at least one of the compressor and the at least one shaft-driven mechanical device is provided. The oxygen-containing gas stream is delivered to the hydrocarbon conversion unit for use in converting the unconverted hydrocarbons.

In still another embodiment, a method is provided for converting light hydrocarbons into heavier hydrocarbons (C5+) with a hydrocarbon conversion system having a gas turbine with a compressor and a combustor, and having a second compressor. The method includes compressing a first oxygen-containing gas stream with the compressor of the gas turbine to provide to the combustor to produce a first compressed oxygen-containing gas feed stream. A second oxygen-containing gas stream is compressed with the second compressor driven by power from the gas turbine to produce a second compressed oxygen-containing gas feed stream. The first compressed oxygen-containing gas feed stream and the second compressed oxygen-containing gas feed stream are delivered to a hydrocarbon conversion unit, wherein the first compressed oxygen-containing gas feed stream is from about 15% to about 35% by volume of the compressed oxygen-containing gas required by the hydrocarbon conversion unit and the second oxygen-containing gas feed stream makes up from about 65% to about 85% by volume of the compressed oxygen-containing gas required by the hydrocarbon conversion unit. Light hydrocarbons are delivered to the hydrocarbon conversion unit to produce heavier hydrocarbons (C5+) and a tail gas in the hydrocarbon conversion unit. The tail gas is delivered to the combustor of the gas turbine.

In another embodiment, a system for converting light hydrocarbons into heavier hydrocarbons is provided. The system includes a gas turbine having a first compressor, combustor, and expander. The gas turbine is operable to produce a first compressed oxygen-containing feed stream and operable to produce additional power. A second compressor is coupled to the gas turbine for receiving power therefrom and operable to produce a second compressed oxygen-containing gas feed stream. A hydrocarbon conversion unit is fluidly coupled to the gas turbine and the second compressor for receiving the first compressed oxygen-containing gas feed stream and the second oxygen-containing gas feed stream. The hydrocarbon conversion system is operable to receive the first and second oxygen-containing gas feed streams, light hydrocarbons, and steam and produces heavier hydrocarbons and a tail gas. A conduit for receiving tail gas from the hydrocarbon conversion unit is provided and delivers the tail gas to the combustor of the gas turbine.

A method for supplying compressed air to a hydrocarbon conversion system is also provided. The method includes compressing air in a compressor of a gas turbine to make from about 15 to about 35 percent by volume of the needed air for the hydrocarbon conversion system. Air is compressed in a second compressor that is coupled to the gas turbine to receive power therefrom to make the remaining 85 to 65 percent by volume of the needed air for the hydrocarbon conversion system. The method may further include supplying a low-BTU tail gas from the hydrocarbon conversion system to a combustor of the gas turbine.

In another embodiment of the invention, a method for converting hydrocarbons in a hydrocarbon conversion system is provided by providing a gas turbine having a combustor, an expander and a compressor. At least one other compressor is coupled to and powered at least in part by the gas turbine on a single drive train. A second power unit, which may include a steam turbine or an electrical motor-generator, is coupled to the at least one other compressor and gas turbine to provide power to the drive train. A gas, which may be an oxygen-containing gas or a syngas, is compressed utilizing the at least one other compressor and is delivered from the at least one other compressor along with unconverted hydrocarbons to a hydrocarbon conversion unit. Hydrocarbon products and tail gas are produced from the unconverted hydrocarbons and compressed gas utilizing the conversion unit.

### BRIEF DESCRIPTION OF THE DRAWINGS

For a more complete understanding of the present invention, and the advantages thereof, reference is now made to the following descriptions taken in conjunction with the accompanying figures, in which:

FIG. 1 is a schematic diagram of one embodiment of a hydrocarbon conversion system according to the present invention;

FIG. 2 is another a schematic diagram of an embodiment of a hydrocarbon conversion system according to the present invention;

FIG. 3A is a schematic diagram of another embodiment of a hydrocarbon conversion system employing supplemental compressors according to the invention;

FIG. 3B is a schematic diagram of an embodiment of a hydrocarbon conversion system employing supplemental compressors and additional shaft-driven devices, in accordance with the invention;

FIG. 3C is a schematic diagram of still another embodiment of a hydrocarbon conversion system employing two supplemental compressors and a gas stream diverted from a compressor of a gas turbine, in accordance with the invention;

FIG. 4A is a schematic diagram of an embodiment of a hydrocarbon conversion system employing a gas turbine and a steam turbine and employing supplemental compressors in accordance with the invention;

FIG. 4B is a schematic diagram of another embodiment of a hydrocarbon conversion system employing a gas turbine and a steam turbine;

FIG. 5 is a schematic diagram of still another embodiment of two parallel hydrocarbon conversion systems, each employing a gas turbine and an electrical motor-generator;

FIG. 6 is a schematic diagram of still another embodiment of a hydrocarbon conversion system utilizing two gas turbines, each employing two supplemental compressors and a steam turbine, with the supplemental compressors of one gas turbine being used to compress and oxygen-containing gas, and the supplemental compressors of the other being used to compress syngas; and

FIG. 7 is a schematic diagram of another embodiment of a hydrocarbon conversion system having a gas turbine having two supplemental compressors and a steam turbine, with one compressor being used to compress an oxygen-containing gas, and the other being used to compress syngas.

### DETAILED DESCRIPTION

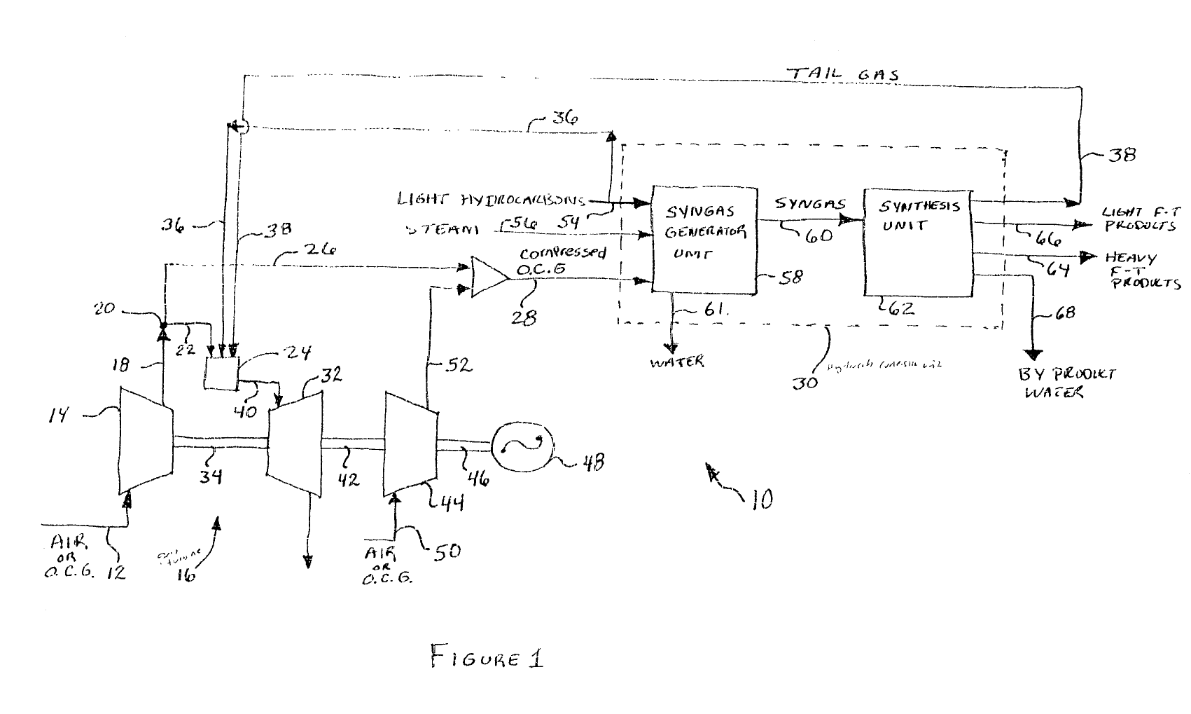

Referring to FIG. 1, a hydrocarbon conversion system 10 according to one embodiment of the present invention is presented. An oxygen-containing gas (OCG), such as air or enriched air, is delivered to a conduit 12 which delivers the gas to a first compressor 14. As used herein, “oxygen-containing gas” shall mean a gas or gas mixture made up of or containing the diatomic form of oxygen or O₂. The oxygen-containing gas may have an oxygen content of from about 1% to about 35% by volume, with from about 10% to 35% by volume being more typical. Unless otherwise indicated, all percentages are given by volume. It may be preferable in many instances to use air as the oxygen-containing gas, due to its ready availability as atmospheric gases. Air is composed primarily of nitrogen and oxygen, and typically has an oxygen-gas (O₂) content of from about 20% to 22% by volume, more typically around 21%, with nitrogen gas (N₂) making up from about 77% to about 79% by volume, more typically around 78%.

The compressor 14 is the compressor for a gas turbine 16. The compressed oxygen-containing gas from first compressor 14 is delivered to an outlet 18. At a junction 20, a first portion of the compressed oxygen-containing gas is delivered through a conduit 22 to a combustor 24 and another portion is delivered as a first compressed oxygen-containing gas feed stream through conduit 26 to compressed oxygen-containing gas inlet 28 of a hydrocarbon conversion unit 30 for use in a conversion process.

The gas turbine 16 includes compressor 14, combustor 24, and an expander 32. The expander 32 is mechanically coupled by a shaft 34 to compressor 14. The combustor 24 receives compressed oxygen-containing gas through conduit 22 and receives a combustion fuel through conduit 36 and/or conduit 38. The resultant combustion gases are delivered through a conduit 40 to the expander 32 where the resultant power drives shaft 34 to compress air with compressor 14. In addition, the expander 32 may drive the same or a second shaft 42 or other means by which power may be coupled to a second oxygen-containing gas compressor 44 and preferably is also coupled by another portion of the shaft 46 or separate shaft or other means of coupling power to a generator 48. The second compressor may be an axial-type or centrifugal compressor. As defined herein, “axial compressor” shall be construed to mean an axial compressor and/or an axial-radial compressor.

The second compressor 44 receives an oxygen-containing gas, such as air or enriched air, through an inlet 50 and compresses the gas to produce a second compressed oxygen-containing gas feed stream, which is delivered by a conduit 52 to the compressed oxygen-containing gas inlet 28 of the hydrocarbon conversion unit 30.

The hydrocarbon conversion unit 30 receives light hydrocarbons, e.g., natural gas, through conduit 54. Conduit 54 may have various preparation devices (such as for filtering, removing sulfur, or heating) included on it to prepare the gas for delivery to unit 30. Steam is delivered through an inlet 56 to the hydrocarbon conversion unit 30. These streams are delivered to a synthesis gas (“syngas”) generator unit 58 of the hydrocarbon conversion unit 30. The syngas generator unit 58 produces syngas which is delivered through conduit 60 to a synthesis unit 62. The synthesis gas generator unit 58 is preferably an autothermal reformer reactor, and the synthesis unit 62 is preferably a Fischer-Tropsch unit. The syngas generator 58 also produces water that is delivered to a conduit 61 from where it is treated and disposed of or used elsewhere in the system 10.

The synthesis unit 62 receives the syngas through conduit 60 and catalytically synthesizes heavier products (preferably made up of mainly C5+products). Synthesis unit 62 produces a heavy product stream (e.g., C18+) that is delivered to a conduit 64 and a light product stream (e.g., C18<liquids) that is delivered to a conduit 66, and a light gaseous residue or tail gas that is delivered to a conduit 38. The tail gas of conduit 38 includes nitrogen and other un-reacted substances. While a large variety of tail gas compositions are possible, an example of a tail gas composition ranges may be as follows: carbon monoxide 3-8%, carbon dioxide 3-8%, hydrogen 3-10%, water 0-0.5%, nitrogen 70-90%, methane 1-7%, ethane 0-1%, propane 0-1%, butane 0-1%, pentane+0-1%, each given in volume percent. A more specific example of a tail gas composition may be as follows: carbon monoxide 4.41%; carbon dioxide 4.49%; hydrogen 8.49%; water 0.06%; nitrogen 79.03%; methane 2.74%; ethane 0.13%; propane 0.28%; butane 0.14%; and pentane+0.23%, all by volume. The tail gas is typically a low-Btu tail gas having a energy value of from about 40 Btu/scf to about 350 Btu/scf, more typically, the tail gas has an energy value of from about 65 Btu/Scf to about 110 Btu/Scf. This is due in large part to the high amounts of nitrogen gas, which constitutes a major component of the tail gas. Typically, nitrogen gas will comprise from 70 to 95% by volume of the tail gas. The combustor 24 may be that specifically designed for combusting a low Btu or low heating value fuel, such as the combustor described in U.S. Pat. No. 6,201,029 to Waycuilus, which is herein incorporated by reference.

The synthesis unit 62 also produces water as a byproduct and contaminates that are delivered to conduit 68 from where it is treated and disposed of or reused elsewhere in the system, such as in the production of steam from the water produced.

In preparing a system like system 10, it is preferable to use a gas turbine 16 that is already manufactured by turbine vendors and commercially available and can be used as is or modified within only minimal alterations to accommodate the system. Efforts have been made to prepare a system like system 10 without the second compressor 44, but it has been found that inadequate amounts of air are pulled off through conduit 26 for use in the conversion unit 30 without significant modifications or redesigns being made to existing turbine designs. Examples of suitable commercially available gas turbines include the GE PG9171E gas turbine, manufactured by General Electric, and the GT11N2 gas turbine, manufactured by Alstom Power, Baden, Switzerland, each with modifications for extraction of air (i.e., conduit 26, etc.), but other models and makers may be used as well.

An important feature of the present invention is that the inclusion of two separate, parallel compressors allows for more air or oxygen-containing gas to be produced. This in turn allows more tail gas to be produced by the conversion unit 30, which is delivered to the combustor 24, and allows more mass flow to the turbine expander 32. Both of these allow more power to be produced by gas turbine 16. The power is used to drive the second compressor 44 as well as—in most instances—generator 48.

The first compressor 14 of gas turbine 16 produces from 0 to 50 percent, preferably, about 15 to about 35 percent of the compressed oxygen-containing gas required by syngas generator 58 of hydrocarbon conversion unit 30. The remaining portion required to meet the need of conversion unit 30 is supplied by second compressor 44, which produces the second oxygen-containing gas feed stream. The first and second oxygen-containing gas feed steams are delivered to the syngas generator unit 58 by inlet 28. In a more preferred embodiment, the first oxygen-containing gas feed stream in conduit 26 makes up about 25 to about 35 percent of the required oxygen-containing gas of syngas generator unit 58 and the remaining 75 to 65 percent is made up by the second compressed oxygen-containing gas feed stream.

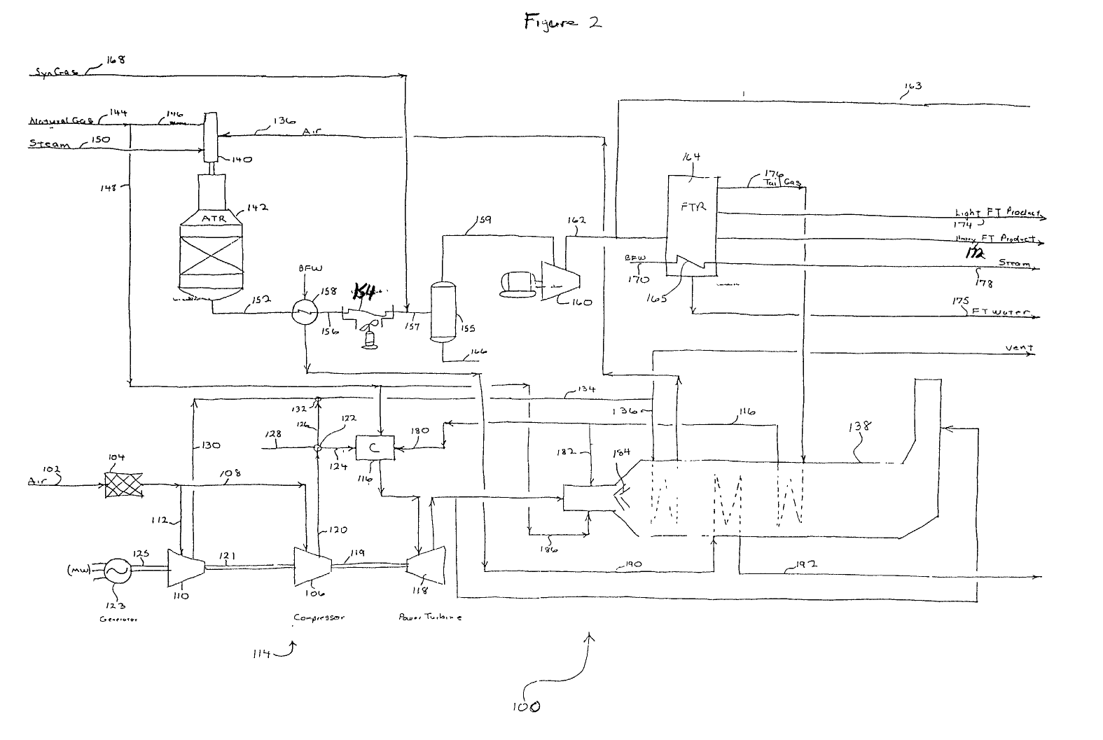

Referring now to FIG. 2, another embodiment of a hydrocarbon conversion system 100 is presented. Air is delivered through an inlet 102 through air filter 104 to a compressor 106 through a conduit 108 and to a second compressor 110 through another conduit 112.

The first compressor 106 is part of a gas turbine 114, which includes the compressor 106, a combustor 116, and a power turbine or expander 118. A shaft 119 mechanically couples the compressor 106 and the power turbine 118. The second compressor 110 is coupled to the gas turbine 114 such that power is delivered from turbine 114 to drive compressor 110. This may be accomplished, for example, with a shaft 121, which may be a continuation of shaft 119 or may be a separate shaft or other means, such as shafts connected by a gear box or clutch mechanism. In addition to powering second compressor 110, the gas turbine 114 preferably has sufficient power to also drive a generator 123, which is coupled to receive power from the gas turbine 114. The generator 123 may be coupled by a shaft or other means as represented by reference number 125.

The compressor 106 receives air, compresses it, and delivers it to an outlet 120 from where it goes to a junction 122. At junction 122, a portion of the compressed air in conduit 120 is delivered through conduit 124 to the combustor 116 and the remaining portion is delivered to conduit 126. The compressed air in conduit 126 constitutes a first compressed oxygen-containing gas feed stream in this embodiment. Conduit 128 may be used to vent compressed air during start-up or stopping of the system 100.

The second compressor 110 produces a second compressed oxygen-containing gas feed stream that is delivered to conduit 130. The feed streams of conduits 126 and 130 are combined at junction 132 and delivered through conduit 134 to conduit 136, which (after passing through heater 138) delivers the compressed air to carburetor 140 of autothermal reformer (ATR) 142 at a pressure of from 50 psig to about 500 psig. The present invention has particular application to those hydrocarbon conversion systems which utilize a low-pressure ATR, i.e. at a pressure below 200 psia, more preferably below 180 psia. In one particular embodiment, the compressed oxygen-containing gas feed is discharged at a pressure of 170 psia, so that it is delivered at 150 psia to the ATR when accounting for pressure losses through process equipment.

Light hydrocarbons, such as natural gas, are delivered through inlet 144 to conduit 146, which in turns delivers them to carburetor 140. The light hydrocarbons or a portion thereof may be delivered through conduit 148 to combustor 116. Steam is delivered through conduit 150 to the carburetor 140. The carburetor 140 delivers the natural gas, steam, and oxygen-containing gas or air to the ATR reactor 142 where syngas is produced and delivered to an outlet 152. The ATR reaction is adiabatic. In other words, no heat is added or removed from the reactor other than from the feeds and the heat of reaction. The reactions that occur are both exothermic and endothermic with the resulting reactor effluent temperature typically ranging from about 500° F. to about 1000° F. above the feed temperature. The effluent syngas exits the reactor in the range of from about 1500° F. to about 3000° F., and is preferably from about 1600° F. to about 2000° F., with a pressure from about 50 to about 500 psig, more preferably from about 100 to 400 psig.

The reactor effluent syngas exits the reactor by conduit 152 and is first cooled by indirect heat exchange such as heat exchanger 158. Heat exchanger 158 may be a series of heat exchangers. These heat exchangers are typically used to generate steam and preheat water for steam production. The high ATR effluent temperature permits the generation of high-pressure steam, which is a valuable source of energy and can be used in many applications throughout the process, including but not limited to the generation of power for driving compressors or electrical power generators.

The syngas is delivered by conduit 156 to air cooler 154 and may be further cooled, typically from about 100° F. to about 130° F. The syngas is then delivered by conduit 157 to a separator vessel 155. Free water produced from the syngas in the cooling process is separated from the syngas by the separator 155 and is delivered to conduit 166. The process water produced is high quality process water and with additional treating it can go to disposal, but is typically used elsewhere in the system 100.

The syngas is delivered by conduit 157 to a syngas booster compressor 160 and then to conduit 162. Conduit 162 delivers the syngas to a Fischer-Tropsch Reactor (FTR) 164. Alternatively, if sufficient pressure exists, the syngas could be delivered without boosting the pressure to the FTR 164. During start-up, syngas may be delivered through conduit 168 to a vent or flare stack.

The FTR is typically maintained at a temperature ranging from about 320° F. to about 600° F. and a pressure from about 300 psig to about 750 psig. The FTR 164 receives syngas through conduit 162. Unlike the ATR, the FTR is not adiabatic. The temperature is controlled in the desired range by removal of heat generated by the Fischer-Tropsch reactions. The heat is typically removed by steam generation within the reactor. Boiler feed water (BFW) is typically delivered to a heat transfer coil 165, which is contained within the reaction zone to remove the heat of reaction and control the FTR temperature. The heat transferred through the coil generates steam which is delivered by conduit 178 and may be used in other areas of the plant.

The FTR produces a number of product streams, which may be represented in various ways. It is understood that the FTR 164 is not a single vessel, but a system that includes a variety of process equipment for cooling and separation of the reactor effluent into the products described. The products shown here include heavy Fischer-Tropsch product stream 172 delivered to outlet 172, a light Fischer-Tropsch stream delivered to outlet 174, a residual gas or tail gas stream delivered to conduit 176, and a Fischer-Tropsch water stream delivered by conduit 175.

The tail gas delivered to conduit 176 is similar to that previously described in connection FIG. 1 and passes through heater 138. After heater 138, a portion of the tail gas is delivered by conduit 180 to the combustor 116 and another portion is delivered through conduit 182 to a burner 184 in heater 138 for use as fuel. During start-up primarily, light hydrocarbons, such as natural gas, may be supplied as fuel to burner 184 through conduit 186. Conduit 190 is representative of other streams, such as streams 166 and 178, containing water or steam that may be delivered to heater 138 to produce super heated steam which is shown exiting through conduit 192, and which may be used in other areas. Conduit 163 is a synthesis gas vent for using during startup and shut-down. An example of a conversion unit or system used in a GTL conversion process that can incorporate the aspects of the present invention is described in U.S. Pat. No. 4,833,170, which is herein incorporated by reference. In this particular system and conversion process air is used as the oxygen-containing gas.

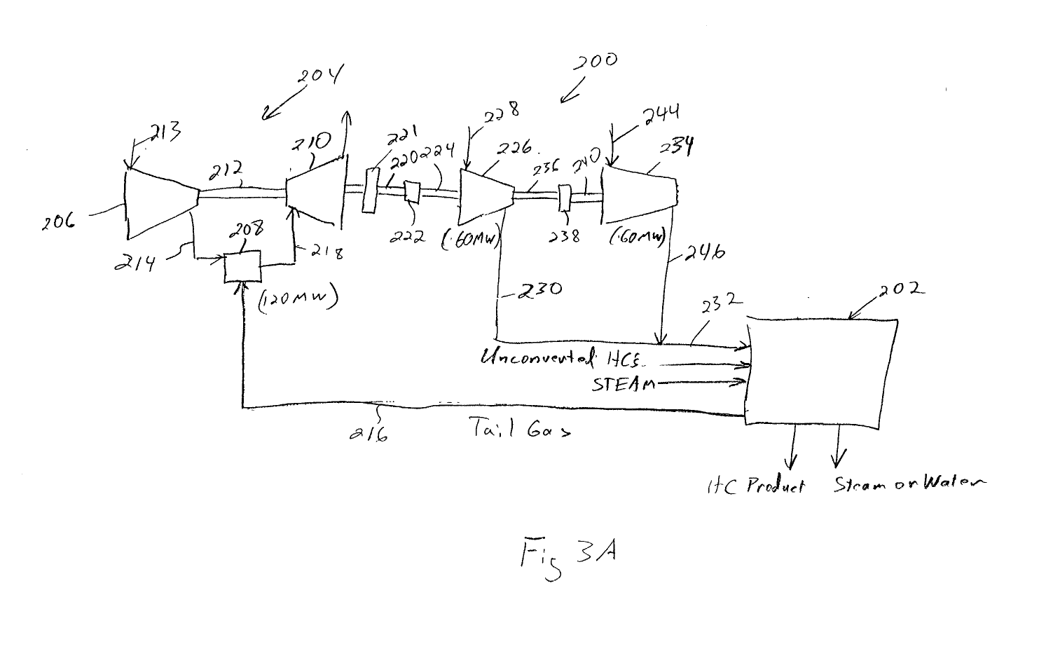

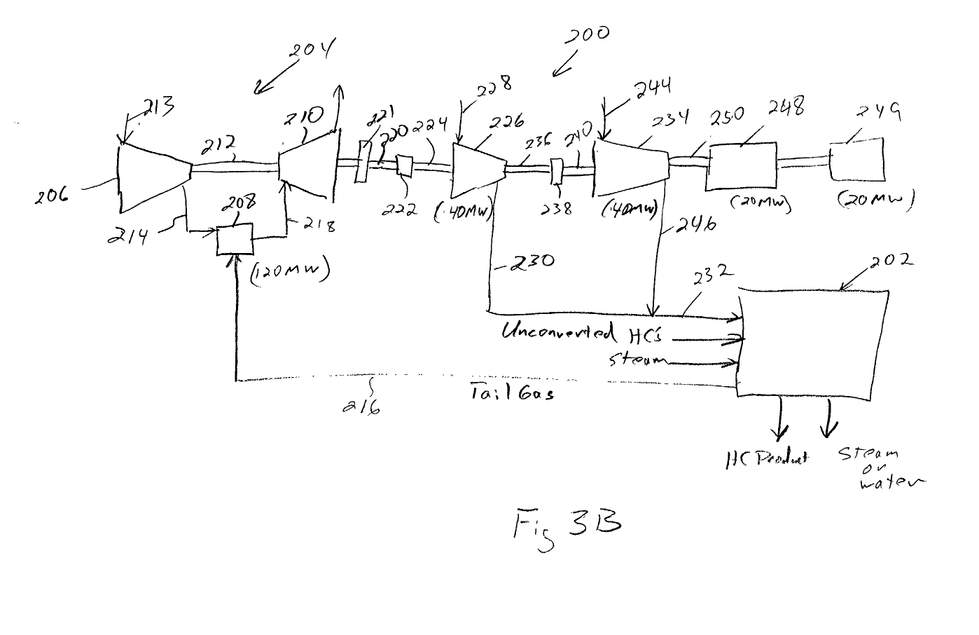

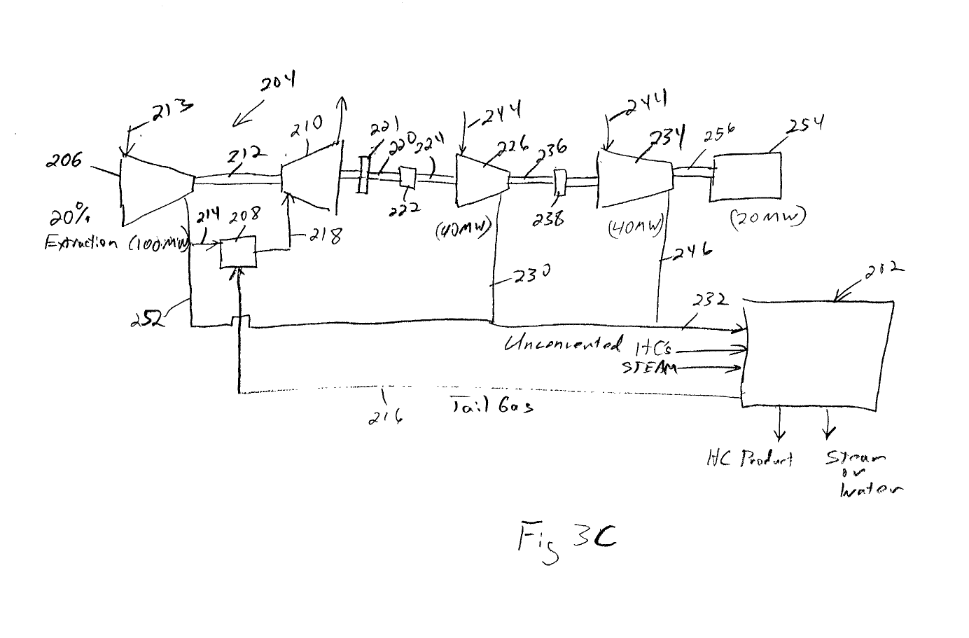

FIG. 3A shows another example of a hydrocarbon conversion system 200 constructed in accordance with the invention. The system 200 includes one or more hydrocarbon conversion units represented at 202, and which may be similar to hydrocarbon units of FIGS. 1 and 2, previously described. A single shaft gas turbine 204 having a compressor 206, a combustor 208 and an expander 210 is provided with the system 200. The turbine 204 is preferably a commercially available gas turbine, such as the GE PG9171 E gas turbine, available from General Electric. Other commercially available gas turbines may be used as well. The expander 210 is coupled to the compressor 206 by a shaft 212, or is otherwise provided with means for driving the compressor 206. It should be noted that various valves and other process equipment that typically would be included in such systems, and would be well within the knowledge of those skilled in the art, are not shown for ease of description purposes.

The combustor 208, which may be a low-Btu-fuel combustor, such as that described in U.S. Pat. No. 6,201,029, receives compressed oxygen-containing gas or air through conduit 214 and receives a combustion fuel of low-Btu tail gas from the conversion unit 202 through conduit 216. The resultant combustion gases are delivered through a conduit 218 to the expander 210 where the resultant power drives rotatable shaft 212 to compress air with compressor 206, which receives oxygen-containing gas or air through inlet 213. In addition, the expander 210 may drive the same shaft or a second rotatable shaft 220. A gear box 221 may be provided to accommodate differences in shaft speeds. In this way, the gas turbine can operate at higher or lower rpm's from other shaft driven equipment, such as the supplemental compressors described below.

As is shown in FIG. 3A, a hydraulic clutch or torque converter 222 is coupled to the shaft 220 and engages rotatable shaft 224, which drives a supplemental compressor 226. The torque converter 222 may be that such as described in the article entitled “Lockup Torque Converter to Assist Compressor Start-Up,” from Diesel & Gas Turbine Worldwide, May 2001, beginning on page 30, which is herein incorporated by reference in its entirety. As is described therein, the converter includes an impeller that would be coupled to the shaft 220 that is driven by the gas turbine 204. The converter also includes a turbine wheel having adjustable guide vanes to control the hydraulic fluid. During startup, the converter is initially drained of hydraulic fluid or oil. As the gas turbine comes to speed, the shaft 220 is rotated, thereby rotating the impeller of the converter. The impeller acts on the hydraulic fluid to drive the turbine wheel so that the shaft 224 is rotated to thus accelerate the compressor 226. The guide vanes of the converter are adjusted to adjust the rotation speed of the shaft 224 or compressor 226 so that it matches that of the shaft 220. Once the speeds are matched, a geared coupling is engaged so that the shaft 220 is mechanically locked with the shaft 224. The converter 222 can then be drained of oil to maximize efficiency.

The torque converter 222 facilitates start up of the compressor 226, as well as other shaft driven devices that may be driven by the gas turbine 204, that would otherwise overwhelm the gas turbine due to inadequate torque during startup required to accelerate the compressor 226 from a standstill or low power state. An example of a suitable commercially available torque converter is the VOSYCON CSTC, available from Voith Turbo GmbH, Crailsheim, Germany.

The first supplemental compressor 226 receives an oxygen-containing gas, such as air or enriched air, through an inlet 228 and compresses the gas to produce a compressed oxygen-containing gas feed stream, which is delivered via conduit 230 to a compressed oxygen-containing gas inlet 232 of the hydrocarbon conversion unit 202.

A second supplemental compressor 234 is also driven by the gas turbine 204. The compressor 234 is coupled to the shaft 224 or another shaft 236 coupled thereto. A torque converter 238 is coupled to the shaft 236 and engages the shaft 240 which engages and drives the compressor 234. The torque converter 238 is similar to the converter 222, previously described, and facilitates startup of the compressor 234 from a stopped or low power state.

The second supplemental compressor 234 receives an oxygen-containing gas, such as air or enriched air, through an inlet 244 and compresses the gas or air to produce a third compressed oxygen-containing gas feed stream, which is discharged and delivered via conduit 246 to the compressed oxygen-containing gas inlet 232 of the hydrocarbon conversion unit 202.

In the particular embodiment shown in FIG. 3A, the gas turbine is shown as being a 120 MW gas turbine. This is used for purposes of example only, as gas turbines of varying power ratings could be used depending upon process or system requirements. The compressors 226 and 234 constitute single shaft axial compressors which are shown as each being rated at 60 MW each.

FIG. 3B shows the hydrocarbon conversion of FIG. 3A, with like components designated with the same reference numbers, but with one or more shaft-driven mechanical devices indicated at 248, 249 that may also be driven by the gas turbine 204. The devices 248, 249 are driven by the shaft 240 or another shaft 250 coupled thereto. A torque converter(s) (not shown) may also be used, if necessary, to facilitate startup of the devices 248, 249. The devices 248, 249 may constitute compressors for producing compressed gas, such as an oxygen-containing gas or air, or it may be used to compress syngas produced in and used in the conversion process or other gases used in the process. The devices 248, 249 may each be an axial compressor, such as a single-shaft axial compressor or axial-radial compressor, or a centrifugal compressor, particularly where higher pressures are desired, or a blower or other gas or air handling shaft-driven equipment. The mechanical devices 248, 249 may also be pumps for pumping liquids used in the process. Additionally, one or more of the devices 248, 249 may constitute a shaft-driven electrical device, such as a generator for producing electricity. Inclusion of additional mechanical or electrical devices driven by the gas turbine 204 will reduce the power supplied to the supplemental compressors 226 and 234 and thus must be taken into account to ensure that the compressed oxygen-containing gas supplied to the conversion unit 202 is sufficient for the conversion process. Thus, in FIG. 3B, the compressors 226, 234 are shown as 40 MW compressors, with the mechanical devices 248, 249 each being 20 MW devices.

FIG. 3C shows a variation of the hydrocarbon conversion system of FIG. 3A, with like components being designated with the same reference numerals. In the system of FIG. 3C, a portion of the compressed oxygen-containing gas or air from the turbine compressor 206 is diverted via conduit 252 and delivered to the oxygen-gas containing inlet 232 of hydrocarbon conversion unit 202. The amount of compressed gas diverted from the turbine may be from 0% to about 50% by volume, with the diverted gas typically ranging from 0 to 35% by volume.

The diversion of compressed gas is shown as reducing somewhat the power output of the gas turbine. This may be the case in certain gas turbine designs where the mass flow to the burner constitutes a limiting factor of the gas turbine. In cases where the mass flow to the burner is at its maximum or upper limit, the diversion of air or oxygen-containing gas will reduce the power of the gas turbine. In the example of FIG. 3C, which shows a 20% diversion of the compressed oxygen-containing gas, the power of the gas turbine 204 may be reduced, in this case from about 120 MW, without diversion, to about 100 MW. The power available for the supplemental compressors 226, 234 are thus shown as each being 40 MW compressors. Additionally, a 20 MW shaft-driven mechanical device 254 is also shown being driven by the turbine 204 coupled to compressor 234 through shaft 256. The device 254 may also be a shaft-driven electrical device, such as an electrical generator or motor-generator.

The power output of the turbine is dependent mainly upon the mass flow provided to the burner and the presence or lack of adequate combustion air. In most gas turbines that utilize conventional high-BTU fuels, the mass flow provided to the burner comes primarily from the compressed air from the compressor. Thus, less air supplied by the compressor results in less mass flow and less combustion, and therefore there is less power output by the gas turbine.

In the present invention, however, the low-BTU tail gas supplied to the gas turbine has a much higher mass flow due to the large amounts of nitrogen present in the low-BTU tail gas. In gas turbines where there is no mass flow limiting factor for the burner of the gas turbine, the large amounts of nitrogen makes up for any lack of mass flow from the compressor and actually allows the gas turbine to “overdrive” the gas turbine, producing from 20% to 30% or more power than the iso-condition nameplate rating of the particular gas turbine at any given ambient temperature and pressure conditions, dependent only upon any mechanical limitations of the gas turbine.

Thus, in the present invention, air can be extracted from the gas turbine compressor without significantly reducing the power output. This is also beneficial when the gas turbine is operated at less than optimal conditions, such as at high ambient temperatures and high altitudes (low pressure) where the power outputs of gas turbines can be severely derated. In certain cases, the power output can be derated by as much as 30% under such conditions. And in fact, due to the increased mass flow provided by the low-BTU tail gas, power under such non-optimal conditions can be recovered, plus additional power to raise the capability of the turbine to up to 25% over its rated capabilities can be achieved. Thus, for example, a 120 MW gas turbine derated to 95 MW because of operating conditions can become a 150 MW turbine, regardless of operating conditions.

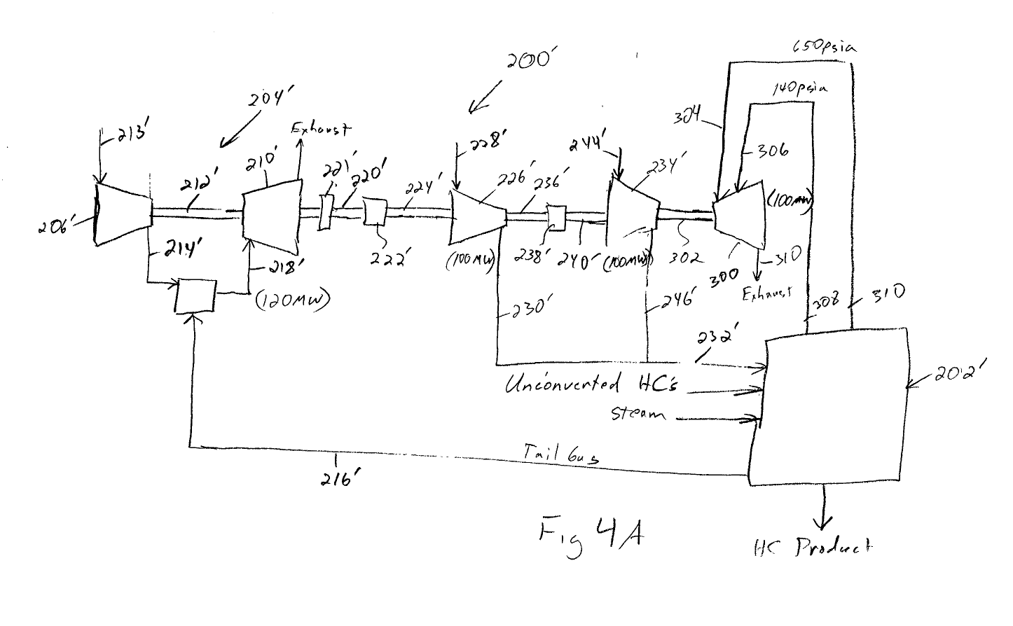

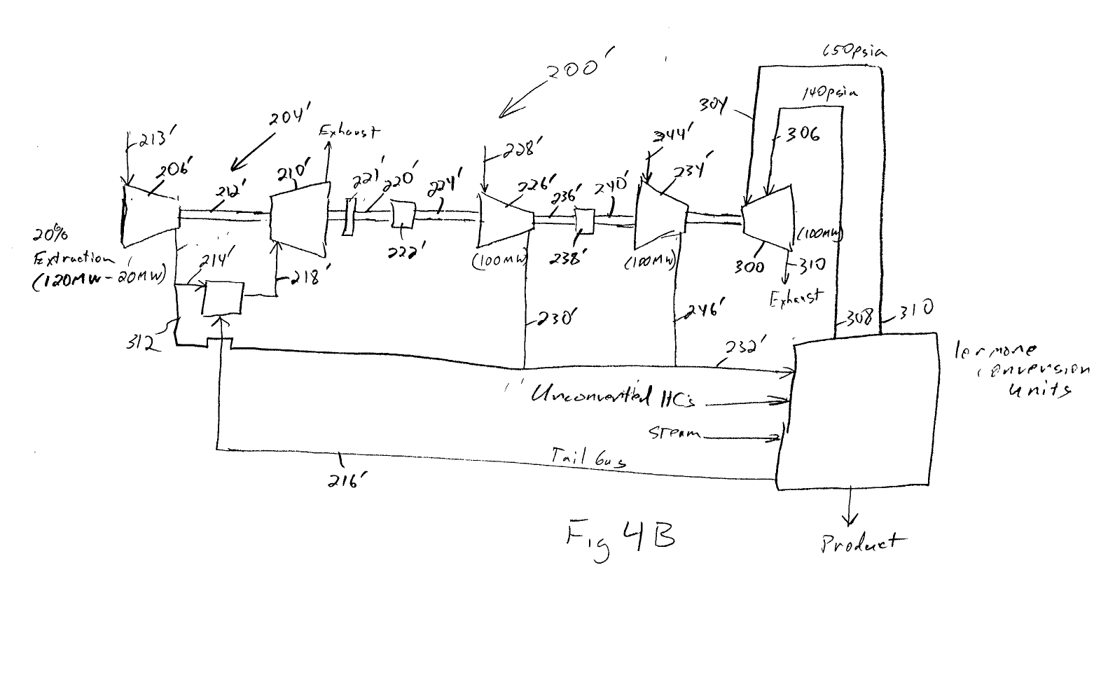

Referring to FIG. 4A, another embodiment of a hydrocarbon conversion system 200′ is shown. The system 200′ is similar to the conversion system 200 of FIGS. 3A-3B, with similar components numbered the same and designated with a prime symbol. The system 200′ includes one or more hydrocarbon conversion units represented at 202′, which are also similar to the hydrocarbon units, previously described. A single shaft gas turbine 204′, such as the GENERAL ELECTRIC PG9171E turbine, having a compressor 206′, a combustor 208′ and an expander 210′ is provided with the system 200′.

The system 200′ is also provided with at least two supplemental compressors 226′ and 234′, which may be driven by the gas turbine 204′ through the same shaft or a series of interconnected shafts, such as the shafts 220′, 224′, 236′, 240′. These are preferably connected or coupled together by means of torque converters 222′, 238′, as have been previously described to facilitate startup, as well as to synchronize the rotation of the shafts, as will be discussed.

Provided with the conversion system 200′ is a steam turbine 300. The steam turbine 300 is coupled to a shaft or series of shafts 220′, 224′, 236′, 240′ driven by the gas turbine 200′. In the embodiment shown, the turbine 300 is coupled to the compressor 234′ through shaft 302. Preferably, disposed between the steam turbine 300 and gas turbine 204′ is one or more clutches or torque converters, such as the torque converters 222′, 238′, previously described. The clutches or torque converters allow for differences in rotations speeds between the shafts driven by the steam turbine 300 and the gas turbine 204′, thus allowing the rotation of the shafts to be synchronized so that the they can be locked together to act as a unitary system. This also allows the compressors 226′, 234′, as well as other shaft-driven equipment (not shown) coupled to the shafts, to be driven independently by either the gas turbine 204′ or steam turbine 300.

The steam turbine 300 is shown as being a multi-pressure steam turbine, such as those steam turbines manufactured by MAN Turbo, GmbH, Oberhausen, Germany, having at least two or multiple inlets for the introduction of steam. As shown, a high pressure inlet 304 and separate lower pressure inlet 306 are provided. Alternatively, two or more single pressure steam turbines could be used to accommodate different pressure steam feeds.

The inlets 304, 306 are each in fluid communication with steam conduits 308, 310, respectively, to deliver steam produced from the hydrocarbon conversion unit 202′. With reference to the conversion unit of FIG. 2, these may be the water or steam byproduct streams 166, 178, that are withdrawn from the ATR 142 and FTR 164, respectively, or other water or steam produced by the conversion unit 202′ during the conversion process. Steam may be provided from other sources, both internal and external to the conversion process. These streams may be heated further, such as through the conduit 190 passing through the heater 138, which may be heated by the exhaust of the gas turbine, to ensure that the water or steam is converted or delivered as superheated steam before being introduced into the steam turbine 300.

In the embodiment shown in FIG. 4A, the pressurized steam introduced at 306 is shown as being at 140 psia, which is a typical pressure for steam or water withdrawn from the FTR reactor. Steam pressure ranges from the FTR are typically 90 to 200 psia. Similarly, the steam introduced at 304 is shown as being at 650 psia, which is a typical pressure for steam withdrawn from the ATR reaction, which may range from 150 to 750 psia. Spent or exhaust steam is discharged from the turbine 300 through conduit 310.

The exhausted steam, which may be exhausted at a pressure of 50 to 75 psia, for example, may be further utilized in other process, such as to drive a desalinization plant, before being condensed and returned as boiler feed water. Alternatively, the steam can be exhausted and directly condensed for reuse as boiler feed water.

In operation, the gas turbine 204′ is started with the introduction of fuel and a compressed oxygen-containing gas into the combustor 208′. At startup, it may be necessary initially to use natural gas or another fuel, due to the lack of tail gas during startup of the conversion process. The clutches or torque converters allow for the sequential start up of the drive train, which in turn facilitates sequential start up of the conversion system. Thus, during start up, tail gas can be produced at partial load and supplied to the gas turbine, thus allowing the gas turbine to be switched over sooner from more expensive start-up fuels.

In the embodiment of FIG. 4A, the turbine 204′ is shown as a 120 MW turbine and the steam turbine 300 is shown as a 100 MW turbine for a combined power generation of 220 MW. The supplemental compressors 226′, 234′ are each shown as 100 MW compressors, so that the excess power can be used to power additional compressors (not shown), such as a compressor for compressing or boosting syngas, as well as other shaft-driven mechanical devices (not shown). The dual powered system could also be used to power shaft-driven electrical devices in the form of electric generators or motor-generators to produce electrical power.

Referring to FIG. 4B, a variation of the hydrocarbon conversion system 200′ of FIG. 4A is shown, with like components being designated with the same reference numbers. In the system of FIG. 4B, a portion of the compressed oxygen-containing gas or air from the turbine compressor 206′ is diverted via conduit 312 and delivered to the oxygen-gas containing inlet 232′ of hydrocarbon conversion unit 202′. The amount of compressed gas diverted from the turbine may be from 0% to about 50% by volume, with the diverted gas typically ranging from 0 to 35% by volume. In the embodiment shown, 20% by volume of the compressed air or gas is diverted. Thus, the power of the gas turbine 204′ is reduced from approximately 120 MW to about 100 MW, assuming a burner that has mass flow limiting factor, as previously discussed, is employed.

By providing a dual or multi-powered system, such as shown in FIGS. 4A and 4B, a continuous stream or constant flow of compressed air or oxygen-containing gas can be readily supplied to the conversion unit 202′. Thus, variations in compressed air or oxygen-containing gas supply resulting from fluctuations in the power generated by either the gas turbine or steam turbine due are compensated for by the other power generation unit. Such fluctuations may be due to variations in the flow or energy value of the low-Btu tail gas supplied to the gas turbine or in the amount of pressurized steam produced by the conversion unit and supplied to the steam turbine. Additionally, the steam turbine/gas turbine combination provides additional power for driving other shaft-driven equipment or for generating power for uses elsewhere, which would not otherwise be available if only the gas turbine were employed.

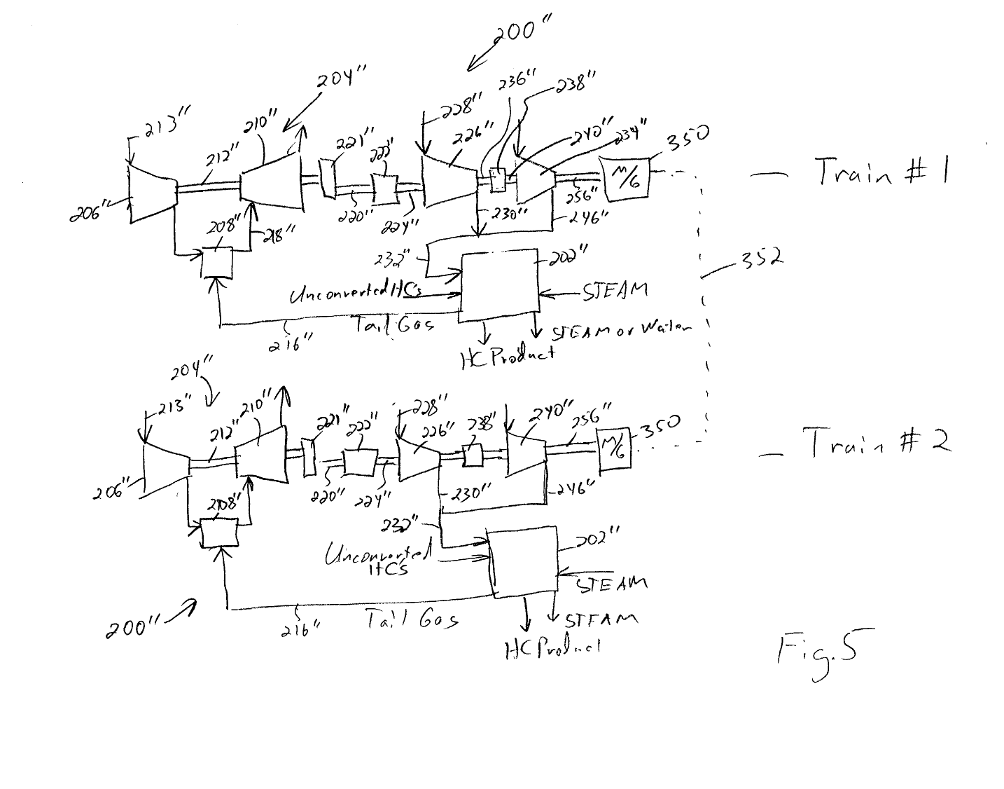

FIG. 5 shows another embodiment involving two conversion systems 200″, with each conversion system 200″ similar to the conversion systems of FIGS. 3A-3C and 4A-4B, with similar components designated with the same numeral and indicated with a double prime symbol. As shown, the two conversion systems 200″ are operated as parallel trains, i.e. Train 1 and Train 2. Operation of the conversion systems 200″ is generally the same as those previously described, however, the drive train of each drives an electrical motor-generator designated at 350 coupled to shaft 256″. The motor-generator 350 can be used selectively as a generator, which is powered by the gas turbine 204″ to produce electrical power to supply to the process or elsewhere. The device 350 can also be operated as a motor when supplied with electrical power to facilitate start up of the gas turbine. Once the gas turbine is operating at high enough capacity, the motor-generator 350 is switched to the generator mode to begin producing electricity.

As shown, the motor-generators 350 of each train are each electrically coupled together at 352. In this way, the motor-generator 350 of one train can be used to facilitate the operation of the other. Thus, for example, after Train 1 has been started and the motor-generator 350 is in generator mode, it can produce electrical power to power the motor 350 of Train 2 to thus facilitate start up of Train 2.

Any number of trains could be coupled together in a similar fashion. Further, if the total drive requirements of the gas turbine of one train were exceeded, a motor-generator from another train could be used to provide power to the motor-generator to the other. Additionally, electrical power could be supplied from an external power source, if necessary.

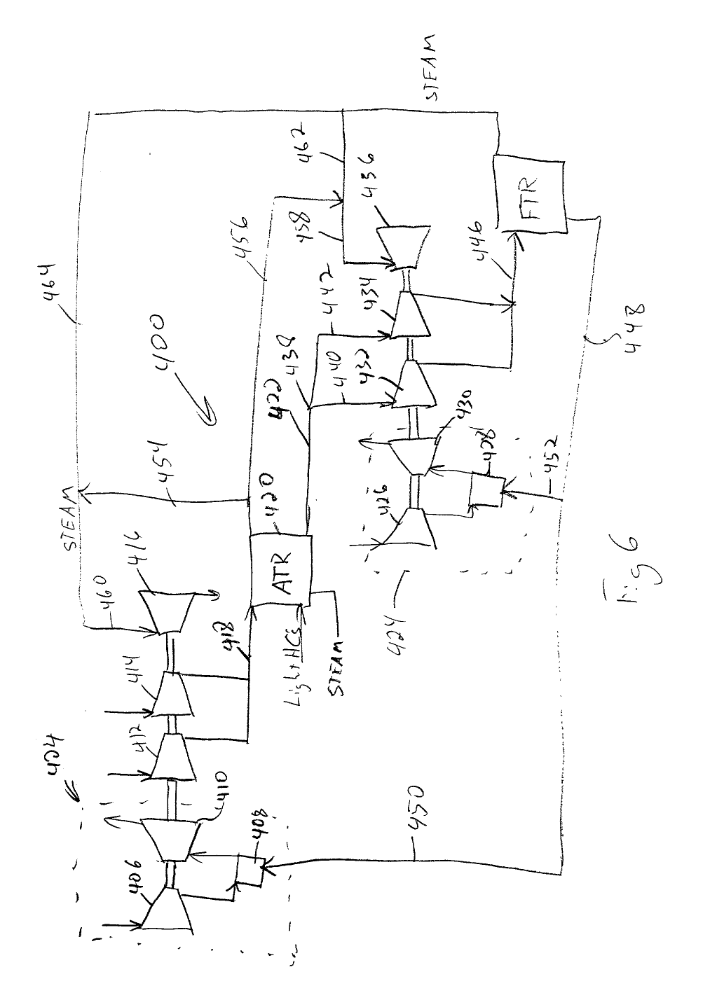

FIG. 6 shows still another embodiment of a hydrocarbon conversion system 400 in accordance with the invention. The conversion system is similar to those previously described. The system 400 is shown simplified, but it should be apparent to those skilled in the art that it would include similar components to those of the above-described conversion systems, such as gear boxes, torque converters, etc.

The conversion system 400 is provided with a gas turbine 404, similar to those previously described, having a compressor 406, a combustor 408 and an expander 410. Coupled to the gas turbine 404 are two shaft-driven compressors for compressing OCG or air. A steam turbine 416 may also be coupled to the compressors and gas turbine on a single drive train for providing additional power and to facilitate start up, if necessary.

In the embodiment shown, compressed air or OCG is provided from the compressors 412 and 414 and is supplied via line 418 to ATR reactor or unit 420, along with unconverted light hydrocarbons and steam or water to produce syngas, which is discharged through line 422. When the OCG is air, such as is used in the GTL conversion process disclosed in U.S. Pat. No. 4,833,170, large amounts of nitrogen gas may be present in the syngas. The nitrogen content of the syngas may range from about 5 to 75% by volume, more typically from about 30 to about 60% by volume, and more typically from about 45 to about 50% by volume.

A second gas turbine 424 is also provided, which may be similar in construction to the turbine 404, and which includes a compressor 426, combustor 428 and expander 430. The turbine 424 is coupled to two shaft-driven syngas booster compressors 432, 434. A steam turbine 436 may also be coupled to the gas turbine 424 and compressors 432, 434 on a single drive train to provide additional power and to facilitate start up.

Syngas from line 422 is split at 438 and delivered to inlets 440, 442 of compressors 432, 434, respectively. The compressors 432, 434 are used to further pressurize or boost the pressure of the syngas for delivery to FTR reactor 444, which operates at higher pressures, via line 446. The syngases are reacted within the FTR, as has been previously described, to produce low-BTU tail gases due to the large nitrogen content, along with converted hydrocarbon products.

The low-BTU tail gases are delivered from the FTR 444 via line 448, and are supplied to burners 408, 428 via lines 450, 452, respectively, for powering the gas turbines.

Process steam from ATR 420 and FTR 444 are delivered to the steam turbines 416, 436, via lines 454, 456, 458, 460, 462 and 464.

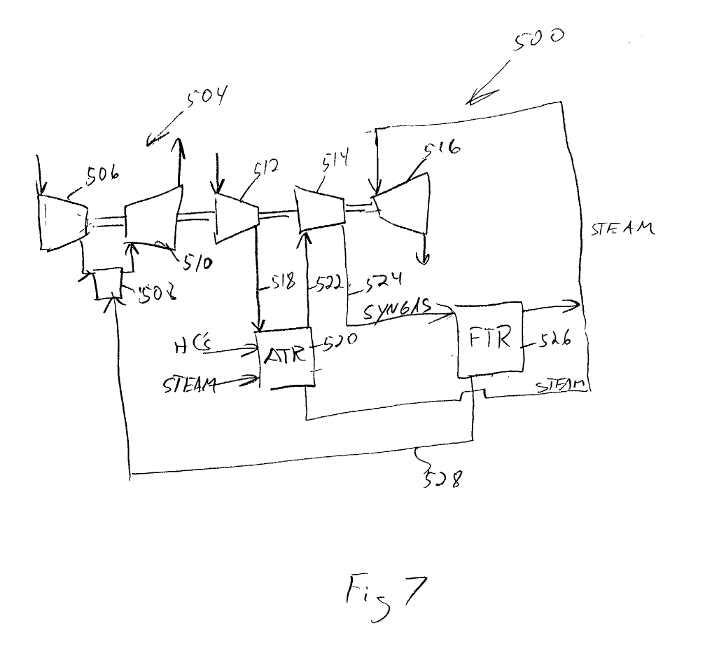

FIG. 7 shows still another embodiment of a hydrocarbon conversion system 500. The conversion system is similar to those previously described and has been simplified for purposes of description. As will be apparent to those skilled in the art, additional components and elements may be incorporated into the system, but are not shown. The system 500 includes a gas turbine 504 having a compressor 506, combustor 508 and expander 510. Coupled to the turbine 504 are shaft-driven compressors 512, 514. A steam turbine 516 may also be coupled to the compressors 512, 514 and turbine 504 on a single drive train.

As shown in FIG. 7, air or OCG is compressed by compressor 512 and delivered via line 518 to ATR 520, where it is reacted in the presence of unconverted hydrocarbons and steam or water to produce syngas. The produced syngas is delivered from ATR 520 to inlet 522 of the second compressor 514, where the syngas is further pressurized or boosted in pressure. The pressurized syngas exits the compressor and is delivered via line 524 to FTR unit 526, where the syngases are converted into converted hydrocarbon products, along with tail gas.

Tail gases from unit 526 are delivered to the combustor 508 via line 528 for powering the turbine 504. Steam from units 520 and 526 are also supplied to the steam turbine 516 via lines 530, 532 and 534.

While the invention has been shown in only some of its forms, it should be apparent to those skilled in the art that it is not so limited, but is susceptible to various changes and modifications without departing from the scope of the invention. Accordingly, it is appropriate that the appended claims be construed broadly and in a manner consistent with the scope of the invention.

We claim:

1. A method for converting hydrocarbons in a hydrocarbon conversion system, the method comprising:
delivering unconverted hydrocarbons to a hydrocarbon conversion unit;
producing hydrocarbon products and tail gas from the unconverted hydrocarbons utilizing the conversion unit;
providing a gas turbine having a combustor, an expander and a compressor;
delivering the tail gas to the gas turbine as fuel to fuel the combustor and power the turbine;
producing a first compressed oxygen-containing gas stream from the compressor of the gas turbine;
providing at least one other compressor that is coupled to and powered at least in part by the gas turbine to produce a second compressed oxygen-containing gas stream;
delivering from about 50% to 100% by volume of the first compressed oxygen-containing gas stream to the combustor and expander, and delivering the remainder of from 0% to about 50% by volume of the first compressed oxygen-containing gas stream to the hydrocarbon conversion unit; and
delivering the second compressed oxygen-containing gas stream to the hydrocarbon conversion unit in an amount of from about 50% to 100% by volume of that required by the hydrocarbon conversion unit, and wherein the first compressor and the at least one other compressor provide all of the compressed oxygen-containing gas required by the conversion unit.

2. The method of claim 1, further comprising:
providing a steam turbine coupled to the at least one other compressor;
delivering pressurized steam to the steam turbine to produce power wherein at least a portion of the power from the steam turbine is provided to the at least one other compressor.

3. The method of claim 1, wherein:
the tail gas is a low-BTU tail gas.

4. The method of claim 1, wherein:
the tail gas is the only fuel delivered to the gas turbine.

5. The method of claim 1, wherein:
the hydrocarbon conversion unit includes a Fischer-Tropsch reactor.

6. The method of claim 1, wherein:
the at least one other compressor is driven by a shaft releasably coupled to the gas turbine.

7. The method of claim 1, wherein:
the at least one other compressor is an axial compressor.

8. The method of claim 1, wherein:
the first compressed oxygen-containing gas feed stream is delivered to the hydrocarbon conversion unit in an amount of from about 15% to about 35% by volume of that required by the hydrocarbon conversion unit; and
the second compressed oxygen-containing gas feed stream is delivered to the hydrocarbon conversion unit in an amount of from about 65% to about 85% by volume of that required by the hydrocarbon conversion unit.

9. The method of claim 1, wherein:
the oxygen-containing gas is air.

10. The method of claim 1, wherein:
the at least one other compressor includes at least two compressors.

11. The method of claim 1,wherein:
the unconverted hydrocarbons are light hydrocarbons of from C₁ to C₄ and the hydrocarbon products are C₅ or higher.

12. The method of claim 3, wherein:
the tail gas provides sufficient mass flow to the gas turbine to compensate for variations in mass flow provided by the first compressed oxygen-containing gas stream delivered to the gas turbine so that power output from the gas turbine is maintained.

13. The method of claim 3, wherein:
the tail gas provides sufficient mass flow to the gas turbine so that power output from the gas turbine is above that of the iso nameplate power output rating of the gas turbine at any given ambient temperature or pressure conditions.

14. A hydrocarbon conversion system, the system comprising:
a gas turbine having a combustor, an expander and a compressor for producing a first compressed oxygen-containing gas stream;
at least one other compressor that is powered at least in part by the gas turbine to produce a second compressed oxygen-containing gas stream;
a hydrocarbon conversion unit for converting unconverted hydrocarbons to converted hydrocarbon products and tail gas, the hydrocarbon conversion unit being in fluid communication with at least one of the turbine compressor and the second compressor for receiving at least one of the first and second compressed oxygen-containing gas streams, the hydrocarbon conversion unit having a tail gas outlet that is in fluid communication with the gas turbine for delivering the tail gas as fuel to the combustor to thereby power the gas turbine.

15. The system of claim 14, further comprising:
a steam turbine coupled to the at least one other compressor, the steam turbine being in fluid communication with a steam outlet of the hydrocarbon conversion unit; and
wherein pressurized steam produced during the conversion of the unconverted hydrocarbons is delivered from the conversion unit to the steam turbine through the steam outlet to produce power, with at least a portion of the power from the steam turbine being provided to the at least one other compressor.

16. The system of claim 14, further comprising:
an electrical motor-generator coupled to the at least one other compressor, the electrical motor-generator being selectively operable as a motor for providing power to the at least one other compressor and as a generator for producing electrical power.

17. The system of claim 14, further comprising:
a releasable coupling for selectively coupling and decoupling the at least one other compressor to the gas turbine.

18. The system of claim 14, wherein:
the hydrocarbon conversion unit produces a low-BTU tail gas that is delivered as fuel to the combustor.

19. The system of claim 14, wherein:
the hydrocarbon conversion unit is a gas-to-liquid (GTL) conversion unit and includes a syngas reactor containing a catalyst for producing a syngas from the unconverted hydrocarbons, and a Fischer-Tropsch reactor containing Fischer-Tropsch catalyst for converting the syngas to liquid hydrocarbons.

20. The system of claim 14, wherein:
the at least one other compressor is a single shaft, axial compressor.

21. The system of claim 14, wherein:
the at least one other compressor includes at least two compressors.

22. A hydrocarbon conversion system comprising:
a gas turbine having a combustor, an expander, and a compressor;
a steam turbine;
a rotatable shaft coupled to the steam turbine and the gas turbine, the shaft being rotatably driven by the gas turbine and steam turbine;
a hydrocarbon conversion unit for converting unconverted hydrocarbons to converted hydrocarbon products and tail gas, the hydrocarbon conversion unit having a tail gas outlet that is in fluid communication with the gas turbine for delivering the tail gas as fuel to the combustor to thereby power the gas turbine, and having a steam outlet in fluid communication with the steam turbine for delivering pressurized steam produced by the hydrocarbon conversion unit during conversion of the unconverted hydrocarbons to the steam turbine to thereby power the steam turbine;
at least one or more shaft-driven mechanical devices that are coupled to the shaft and driven when the shaft is rotated; and
a compressed oxygen-containing feed stream conduit in fluid communication between the hydrocarbon conversion unit and at least one of the compressor and said one or more shaft-driven mechanical devices for delivering compressed air to the hydrocarbon conversion unit.

23. A method for converting hydrocarbons comprising:
converting unconverted hydrocarbons to converted hydrocarbon products and tail gas in a hydrocarbon conversion unit and producing pressurized steam as a byproduct of the conversion process;
delivering the tail gas as fuel to a gas turbine having a combustor, an expander, and a compressor for powering the gas turbine;
delivering the pressurized steam to a steam turbine for powering the steam turbine;
coupling the gas turbine and steam turbine to a rotatable shaft that drives at least one shaft-driven mechanical device during operation of the gas and steam turbines;
providing a compressed oxygen-containing gas stream from at least one of the compressor and the at least one shaft-driven mechanical device; and
delivering the oxygen-containing gas stream to the hydrocarbon conversion unit for use in converting the unconverted hydrocarbons.

24. A method for converting light hydrocarbons into heavier hydrocarbons (C5+) with a hydrocarbon conversion system having a gas turbine with a compressor and a combustor, and having a second compressor, the method comprising:
compressing a first oxygen-containing gas stream with the compressor of the gas turbine to provide to the combustor to produce a first compressed oxygen-containing gas feed stream;
compressing a second oxygen-containing gas stream with the second compressor driven by power from the gas turbine to produce a second compressed oxygen-containing gas feed stream;
delivering the first compressed oxygen-containing gas feed stream and the second compressed oxygen-containing gas feed stream to a hydrocarbon conversion unit, wherein the first compressed oxygen-containing gas feed stream is from about 15% to about 35% by volume of the compressed oxygen-containing gas required by the hydrocarbon conversion unit and the second oxygen-containing gas feed stream makes up from about 65% to about 85% by volume of the compressed oxygen-containing gas required by the hydrocarbon conversion unit;
delivering light hydrocarbons to the hydrocarbon conversion unit;
producing heavier hydrocarbons (C5+) and a tail gas in the hydrocarbon conversion unit; and
delivering the tail gas to the combustor of the gas turbine.

25. A system for converting light hydrocarbons into heavier hydrocarbons, the system comprising:
a gas turbine having a first compressor, combustor, and expander, the gas turbine operable to produce a first compressed oxygen-containing feed stream and operable to produce additional power;
a second compressor coupled to the gas turbine for receiving power therefrom and operable to produce a second compressed oxygen-containing gas feed stream;
a hydrocarbon conversion unit fluidly coupled to the gas turbine and the second compressor for receiving the first compressed oxygen-containing gas feed stream and the second oxygen-containing gas feed stream, the hydrocarbon conversion system operable to receive the first and second oxygen-containing gas feed streams, light hydrocarbons, and steam and produce heavier hydrocarbons and a tail gas; and
a conduit for receiving tail gas from the hydrocarbon conversion unit and delivering the tail gas to the combustor of the gas turbine.

26. A method of supplying compressed air to a hydrocarbon conversion system, the method comprising:
compressing air in a compressor of a gas turbine to make from about 15% to about 35% by volume of the needed air for the hydrocarbon conversion system; and
compressing air in a second compressor that is coupled to the gas turbine to receive power therefrom to make the remaining 85% to 65% by volume of the needed air for the hydrocarbon conversion system.

27. The method of claim 26 wherein the gas turbine includes a combustor and further comprising supplying a low-BTU tail gas to the combustor from the hydrocarbon conversion system.

28. A method for converting hydrocarbons in a hydrocarbon conversion system, the method comprising:
providing a gas turbine having a combustor, an expander and a compressor;
providing at least one other compressor that is coupled to and powered at least in part by the gas turbine on a single drive train;
coupling a second power unit to the at least one other compressor and gas turbine to provide power to the drive train;
compressing a gas utilizing the at least one other compressor;
delivering the compressed gas from the at least one other compressor along with unconverted hydrocarbons to a hydrocarbon conversion unit; and
producing hydrocarbon products and tail gas from the unconverted hydrocarbons and compressed gas utilizing the conversion unit.

29. The method of claim 28, wherein:
the gas is an oxygen-containing gas.

30. The method of claim 28, wherein:
the gas is a syngas.

31. The method of claim 28, wherein:
the second power unit is a steam turbine.

32. The method of claim 28, wherein:
the second power unit is an electrical motor-generator.
